(19) United States
(12) Patent Application Publication
(10) Pub. No.: US 2021/0369949 A1
(43) Pub. Date: Dec. 2, 2021

(54) SYSTEM AND METHOD FOR SUBSTANCE DELIVERY

(71) Applicant: Koglo Ltd., Tel-Aviv (IL)

(72) Inventors: Yotam KAUFMAN, Tel-Aviv (IL); Gilad LAVI, Rishon-LeZion (IL)

(73) Assignee: Koglo Ltd., Tel-Aviv (IL)

(21) Appl. No.: 17/285,120

(22) PCT Filed: Sep. 10, 2019
(86) PCT No.: PCT/IL2019/051014

§ 371 Date: Apr. 14, 2021

Related U.S. Application Data
(60) Provisional application No. 62/746,021, filed on Oct. 16, 2018.

(51) Int. Cl. A61M 5/14

(52) U.S. Cl. CPC A61M 5/1415; A61M 2005/1416; A61M 2250/00

(57) ABSTRACT

A system for delivering a fluid to tissue and a method of using same are provided. The system includes a tissue clip for securing a tissue fold and a tissue puncturing device attachable to the tissue clip. The system further includes a mechanism for sensing the tissue fold and releasing a safety mechanism in the tissue puncturing device so as to enable manual deployment of a tissue puncturing element from the tissue puncturing device.

### RELATED APPLICATION

This application claims the benefit of priority of U.S. Provisional Patent Application No. 62/746,021 filed on 16 Oct. 2018, the contents of which are incorporated herein by reference in their entirety.

### BACKGROUND

The present invention relates to a system for delivering substances into a tissue fold and, more particularly, to a system and method for subcutaneous delivery of an infusion fluid.

Autoinjector devices for delivering a substance into tissue are well known in the art. Such devices are typically used to deliver a medicament into a tissue. Autoinjector devices capable of grasping a tissue are also known in the art.

Autoinjectors are designed for automatic delivery of a substance and as such, autoinjector needle deployment is typically triggered via skin contact or skin fold creation. Such capabilities make autoinjectors unsuitable for infusing a fluid in dehydrated subjects.

Dehydration is a common problem in pets and can cause serious complications and even death.

Pets suffering from dehydration are typically treated via intravenous or subcutaneous fluid infusion. Such infusion can be carried out by the pet owner at home but is typically carried out by a veterinarian in a clinic due to issues of needle phobia and safety associated with presently available infusion devices.

There is thus a need for, and it would be highly advantageous to have, a system for enabling subcutaneous infusion of fluids in a safe and effective manner. Such a system can enable pet owners to treat dehydrated pets at home without the aforementioned limitations of presently used infusion devices.

### SUMMARY

According to one aspect of the present invention there is provided a system for delivering a fluid to tissue comprising a tissue clip for securing a tissue fold; a tissue puncturing device attachable to the tissue clip. The system optionally also includes a mechanism for sensing the tissue fold and releasing a safety mechanism in the tissue puncturing device so as to enable manual deployment of a tissue puncturing element from the tissue puncturing device.

According to further features in preferred embodiments of the invention described below, the tissue clip includes a pair of jaws for trapping the tissue fold at the tissue surface or above it.

According to still further features in the described preferred embodiments the tissue clip is configured such that the pair of jaws are oriented parallel to a tissue surface when the tissue fold is trapped therein. The tissue clip can be configured such that the jaws trap the tissue close to the tissue surface or displaced therefrom. According to still further features in the described preferred embodiments the tissue puncturing device delivers the tissue puncturing element parallel to a surface of the tissue when the tissue clip secures the tissue fold.

According to still further features in the described preferred embodiments the tissue puncturing element is not deployable when the tissue puncturing device is not attached to the clip.

According to still further features in the described preferred embodiments the tissue puncturing element is not deployable when the tissue fold is not secured within the tissue clip.

According to still further features in the described preferred embodiments the tissue puncturing element is deployable out of the tissue puncturing device via a first spring.

According to still further features in the described preferred embodiments the tissue puncturing element is retractable into the tissue puncturing device via a second spring.

According to still further features in the described preferred embodiments the tissue puncturing element is a hypodermic needle.

According to still further features in the described preferred embodiments the tissue puncturing device is connectable to a fluid delivery conduit.

According to still further features in the described preferred embodiments the tissue puncturing element is in fluid communication with the fluid delivery conduit only when in a deployed position.

According to still further features in the described preferred embodiments the mechanism for sensing the tissue fold includes a hinged plate capable of abutting a delivery end of the tissue puncturing device when the tissue fold is present.

According to still further features in the described preferred embodiments the plate covers the delivery end of the tissue puncturing device and includes an opening alignable with a delivery port of the tissue puncturing element.

According to still further features in the described preferred embodiments the tissue puncturing element is only deployable following securement of the tissue fold within the clip and attachment of the tissue puncturing device to the tissue clip.

According to another aspect of the present invention there is provided a method of delivering a fluid into a skin fold comprising securing the skin fold within a tissue clip; attaching a tissue puncturing device to the tissue clip thereby deploying a mechanism for sensing the tissue fold and releasing a safety mechanism in the tissue puncturing device; and deploying a tissue puncturing element from the tissue puncturing device into the skin fold.

According to still further features in the described preferred embodiments the tissue puncturing device is in fluid communication with a source of fluid.

According to still further features in the described preferred embodiments the fluid is saline.

According to still further features in the described preferred embodiments (c) is effected by a manual trigger.

According to still further features in the described preferred embodiments the mechanism for sensing the tissue fold includes a hinged plate capable of abutting a delivery end of the tissue puncturing device when the tissue fold is present.

According to still further features in the described preferred embodiments the plate covers the delivery end of the tissue puncturing device and includes an opening alignable with a delivery port of the tissue puncturing element.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described below. In case of conflict, the patent specification, including definitions, will control. In addition, the materials, methods, and examples are illustrative only and not intended to be limiting.

### BRIEF DESCRIPTION OF THE SEVERAL VIEWS OF THE DRAWINGS

The invention is herein described, by way of example only, with reference to the accompanying drawings. With specific reference now to the drawings in detail, it is stressed that the particulars shown are by way of example and for purposes of illustrative discussion of the preferred embodiments of the present invention only, and are presented in the cause of providing what is believed to be the most useful and readily understood description of the principles and conceptual aspects of the invention. In this regard, no attempt is made to show structural details of the invention in more detail than is necessary for a fundamental understanding of the invention, the description taken with the drawings making apparent to those skilled in the art how the several forms of the invention may be embodied in practice.

In the drawings:

FIG. 1 illustrates one embodiment of the present system showing the tissue puncturing device detached from the tissue clip with the trigger rotationally positioned in the safe position.

FIG. 2 illustrates one embodiment of the present system showing the tissue puncturing device attached to the tissue clip with the trigger rotationally positioned in the armed position.

FIG. 3 illustrates one embodiment of the present system showing the needle deployed from the distal end of the tissue puncturing device.

FIG. 4 illustrates one embodiment of the present system showing trigger rotationally positioned in the needle retraction position.

FIG. 5 illustrates one embodiment of the present system showing retraction of the needle.

FIGS. 6A-C illustrate tissue fold clipping (FIG. 6A), closing of the hinged plate against the tissue fold to release a first safety (FIG. 6B) and delivery of the needle into the tissue fold (FIG. 6C).

FIGS. 7A-C is a side cutaway view of the tissue puncturing device showing the hinged plate in a closed (deployable) position (FIG. 7A) and showing the needle deployed (FIG. 7B) and retracted (FIG. 7C).

FIGS. 7D-F illustrate an embodiment of a locking mechanism following needle deployment.

FIGS. 8A-B illustrate one embodiment of the tissue clip of the present system.

FIGS. 9A-D illustrate the tissue puncturing device elements that protect against accidental needle deployment.

FIG. 10 illustrates infusion of fluids into a cat using of the present system.

FIG. 11 illustrates tissue fold (T) injection site.

FIGS. 12A-B illustrate another embodiment of the present tissue clip, shown separately alongside the tissue puncturing device (FIG. 12A) and assembled with the tissue puncturing device (FIG. 12B).

FIGS. 13A-B illustrate deployment of the needle from the tissue puncturing device positioned within the tissue clip.

FIGS. 14-15 illustrate the tissue clip of FIG. 12A attached to a skin fold on a back of a cat (FIG. 14) and the tissue puncturing device positioned within the skin fold-attached tissue clip (FIG. 15).

FIGS. 16A-B illustrate use of a prototype device shown without (FIG. 16A) and with (FIG. 16B) attached injector.

### DETAILED DESCRIPTION

The present invention is of a system which can be used to deliver a fluid into a tissue. Specifically, the present invention can be used for subcutaneous infusion in dehydrated mammals such as cats.

The principles and operation of the present invention may be better understood with reference to the drawings and accompanying descriptions.

Before explaining at least one embodiment of the invention in detail, it is to be understood that the invention is not limited in its application to the details of construction and the arrangement of the components set forth in the following description or illustrated in the drawings. The invention is capable of other embodiments or of being practiced or carried out in various ways. Also, it is to be understood that the phraseology and terminology employed herein is for the purpose of description and should not be regarded as limiting.

Administering supplemental fluids can benefit dehydrated pets or pets with medical conditions such as kidney disease or chronic renal failure (CRF).

Although such fluid administration can be provided at home using a bag of fluids, a fluid drip set, and a needle, giving injections is outside the comfort zone for most pet owners and as such fluid infusions are typically carried out in a clinical setting.

While reducing the present invention to practice, the present inventor sought out to devise an infusion system that can be safely and easily operated by the pet owner in a home setting while eliminating any apprehensions with needle injections.

Thus, according to one aspect of the present invention there is provided a system for delivering fluids to a subject. The subject can be a pet such as a cat or any mammalian subject in need of subcutaneous injection of a fluid for rehydration or treatment (e.g. a human subject in need of a medicament).

The system includes a tissue clip for creating and securing a tissue fold (e.g. skin fold) from a tissue surface (i.e. folding upward/tenting the surface tissue) and a tissue puncturing device attachable to the tissue clip.

The tissue clip includes jaws for grasping a tissue fold or for securing a tissue fold grasped by the user's fingers. The clip jaws are configured such that they apply a force large enough to secure the tissue fold without causing ischemia and tissue necrosis. The tissue clip preferably lies parallel to the tissue surface such that a tissue fold grasped thereby extends perpendicular to the longitudinal axis of the tissue clip. One configuration of a tissue clip of the present invention is described in more detail below.

The tissue puncturing device includes a deployable tissue puncturing element (e.g. needle) and is attachable to the tissue clip following tissue securement. The tissue puncturing device is attached in an orientation that delivers a tissue puncturing element through a side of the tissue fold. In the case of a skin fold, this ensures that the needle does not penetrate tissue such as muscle tissue disposed below the skin.

As is mentioned herein, one proposed use for the present system is in subcutaneous infusion. In order to ensure proper subcutaneous access prior to fluid delivery, the present system includes two separate deployment safety mechanism, a first mechanism prevents needle deployment if the tissue puncturing device—tissue clips are not attached or are attached incorrectly, the second mechanism prevents needle deployment if a tissue fold is not trapped within the tissue clip or if the trapped tissue fold is not of a correct size.

Once both of these safety mechanisms are released the system can be manually triggered to release the needle into the tissue fold.

In order to further increase the safety of the present system, the tissue puncturing device is configured capable of retracting the needle following fluid delivery. Such retraction is enabled via release of the retraction safety.

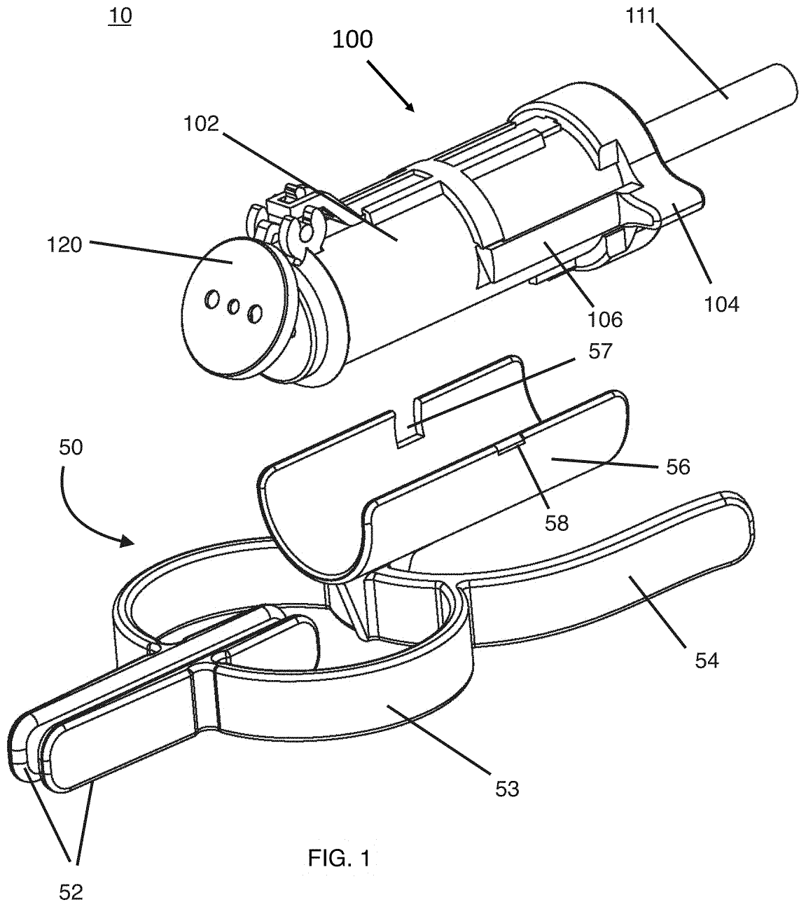

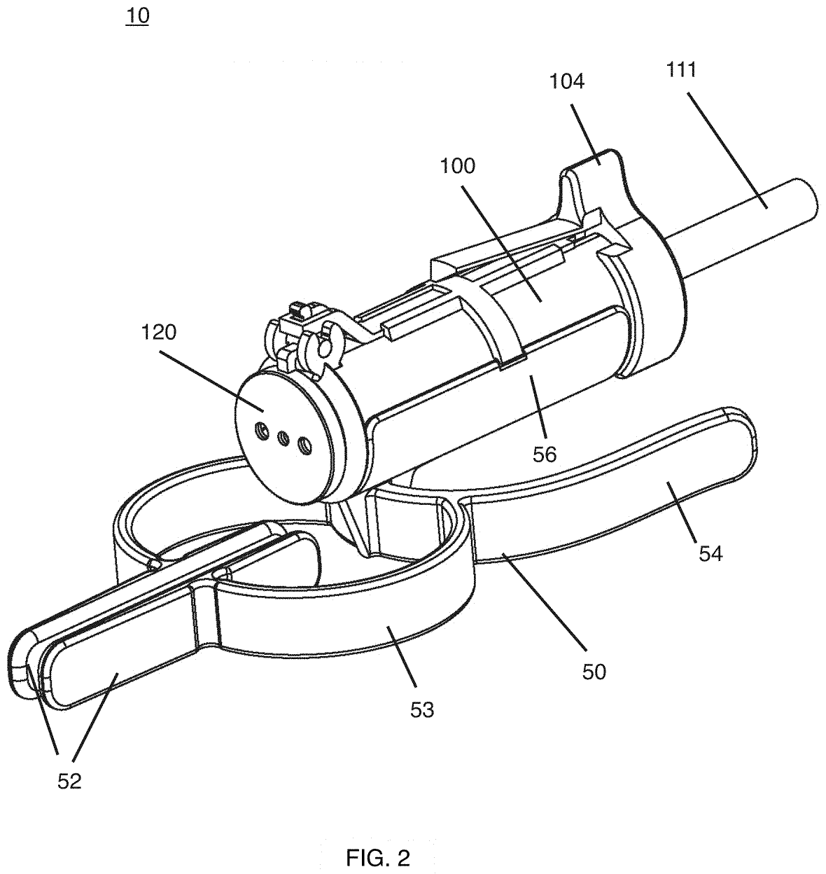

Referring now to the drawings, FIGS. 1-9 illustrate one embodiment of the present system which is referred to herein as system 10.

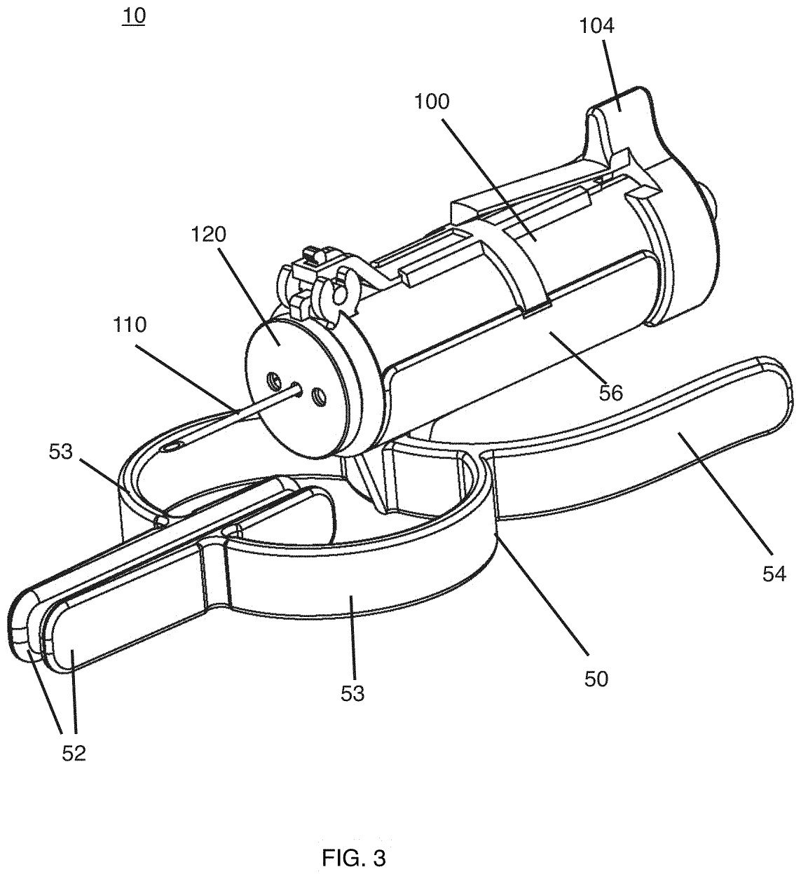

System 10 includes tissue clip 50 and tissue puncturing device 100 shown in the unattached state in FIG. 1 and attached in FIGS. 2-5.

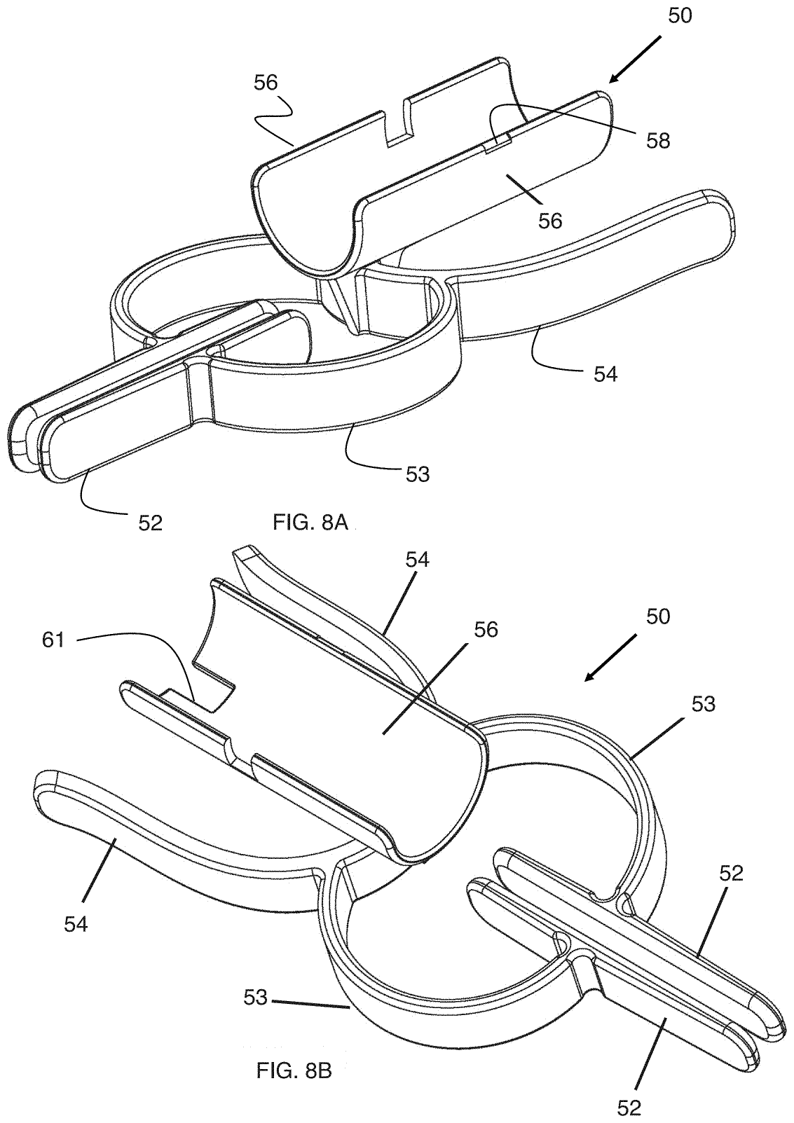

Tissue clip 50 includes jaws 52 that are connected to sprung arms 54 (also shown in FIGS. 8A-B) via semicircular jaw connectors 53. Manually squeezing arms 54 opens jaws 52, release of arms 54 closes jaws 52 and enables grasping of a tissue fold therein. Tissue clip 50 can be fabricated from an elastic polymer such as medical grade Acetal or Polycarbonate or an alloy such as stainless steel.

Tissue clip 50 can be 5-15 cm in length with a maximum opening distance between jaws 52 of 5-7.5 cm. A tissue fold captured by jaws 52 can be 5-10 mm in thickness and 50-150 mm in height (above jaws 52). The force applied by Jaws 52 can be 0.1-1 N/cm².

Tissue clip 50 further includes a cradle 56 for engaging tissue puncturing device 100. Cradle 56 includes cutout 57 and indent 58 for correctly aligning with respective elements on tissue puncturing device 100 and allowing release of a first safety mechanism (FIG. 8A). Cradle 56 also include cutout 61 (FIG. 8B) for preventing tissue puncturing device 100 from rotating when engaged with tissue clip 50 and when trigger 104 is rotated between the operating positions.

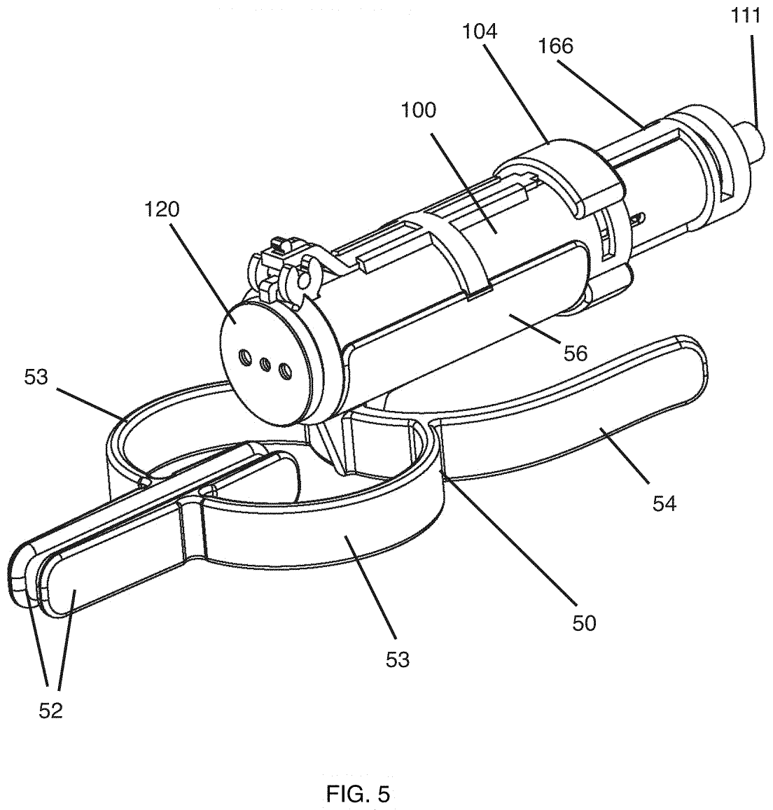

Tissue puncturing device 100 includes a device body 102 having a rotational trigger 104 disposed thereupon. Trigger 104 has three rotational positions, a safe position (shown in FIG. 1), a needle deployment position (shown in FIG. 2) and a needle retraction position (Shown in FIG. 4).

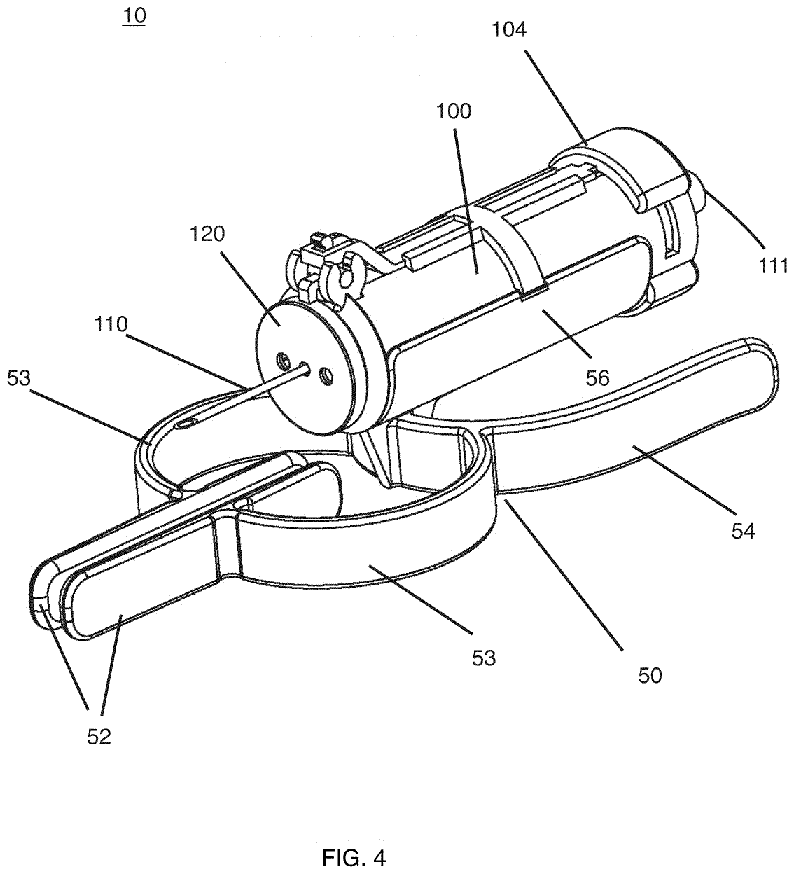

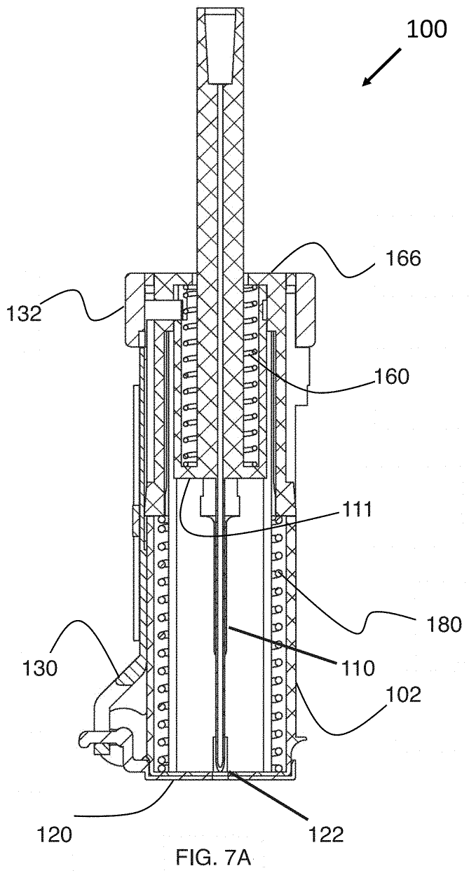

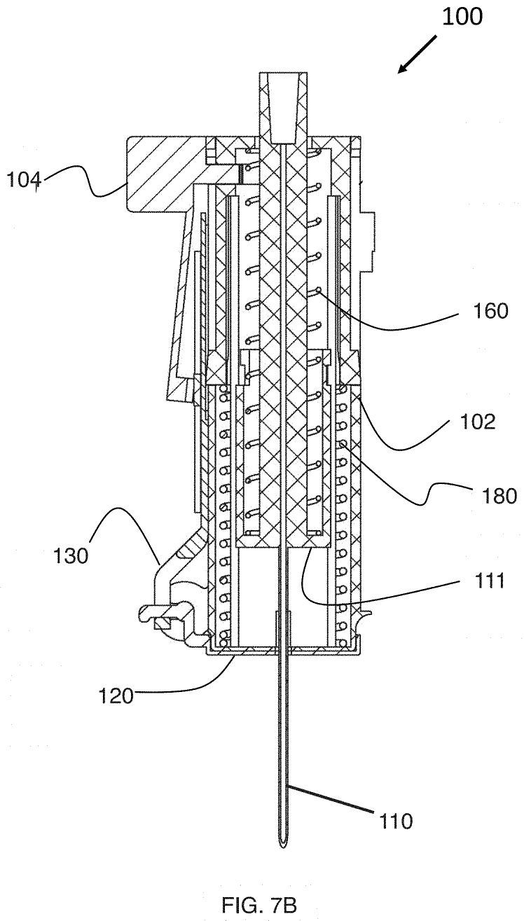

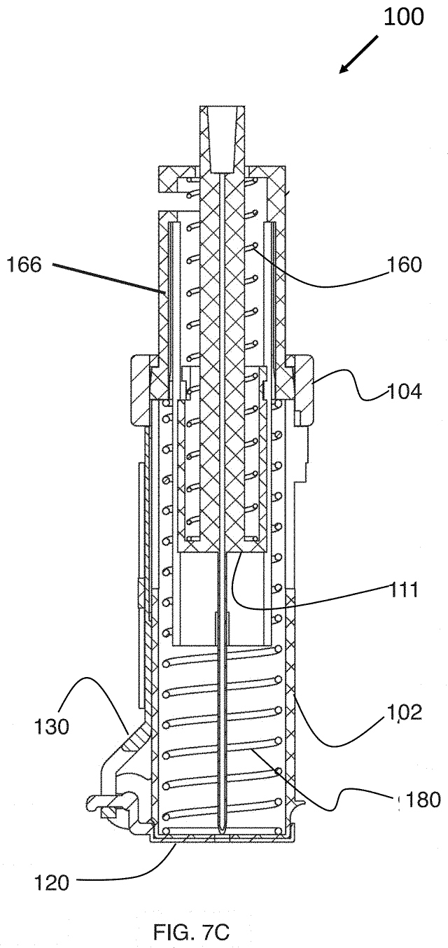

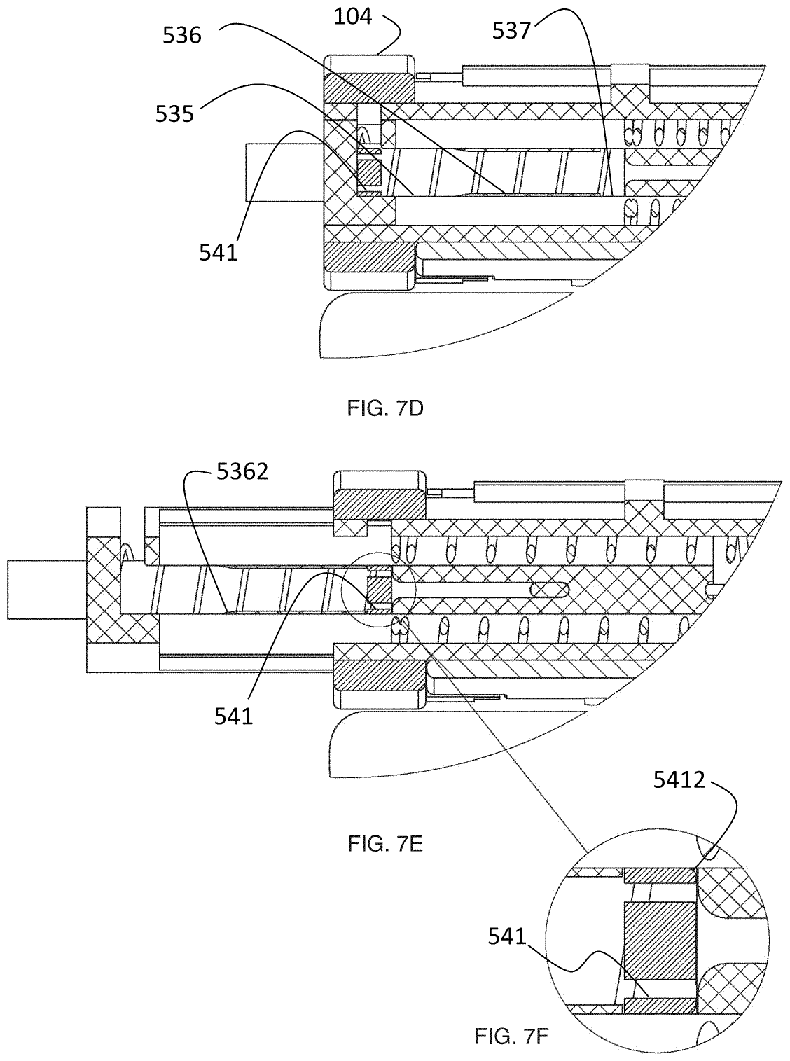

The needle deployment position releases needle 110 (FIG. 3) held under force of a first spring 160 (FIGS. 7A-C).

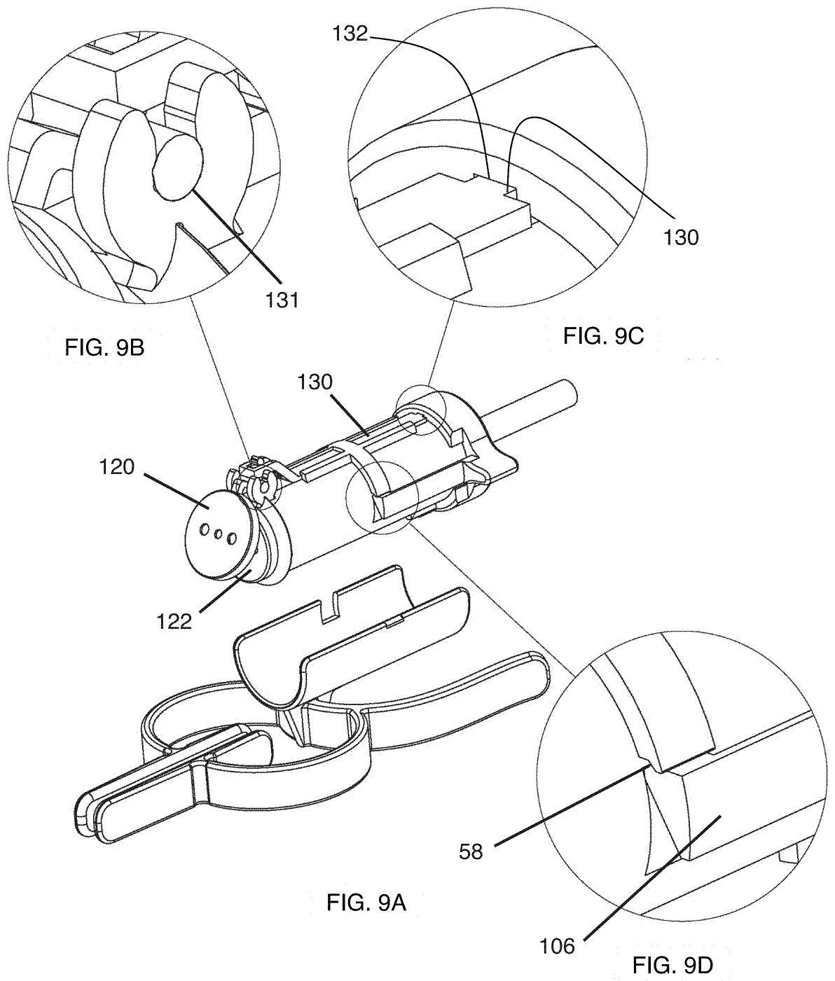

Trigger 104 can be rotated from safe to needle deployment only in the event that tissue puncturing device 100 is correctly engaged within cradle 56. If correctly engaged, cutouts 57 and 61 align with tabs on tissue puncturing device 100 and indent 58 pushes locking arm 106 (FIGS. 1 and 9D) away from device body 102 and enables rotation of trigger 104. Rotation of trigger to the needle retraction position is only enabled once needle 110 is in a deployed position (FIG. 3). Triggering of needle 110 retraction releases a second spring 180 that pushes out (proximally) a cylindrical assembly 166 carrying needle 110 therein (FIGS. 7A-C).

System 10 includes a second safety mechanism that ensures deployment of needle 110 only when a tissue fold of suitable dimensions is grasped within jaws 52.

In the embodiment shown in FIGS. 1-5 and 6A-C, such a mechanism includes a hinged plate 120 capable of abutting a delivery end 122 of tissue puncturing device 100.

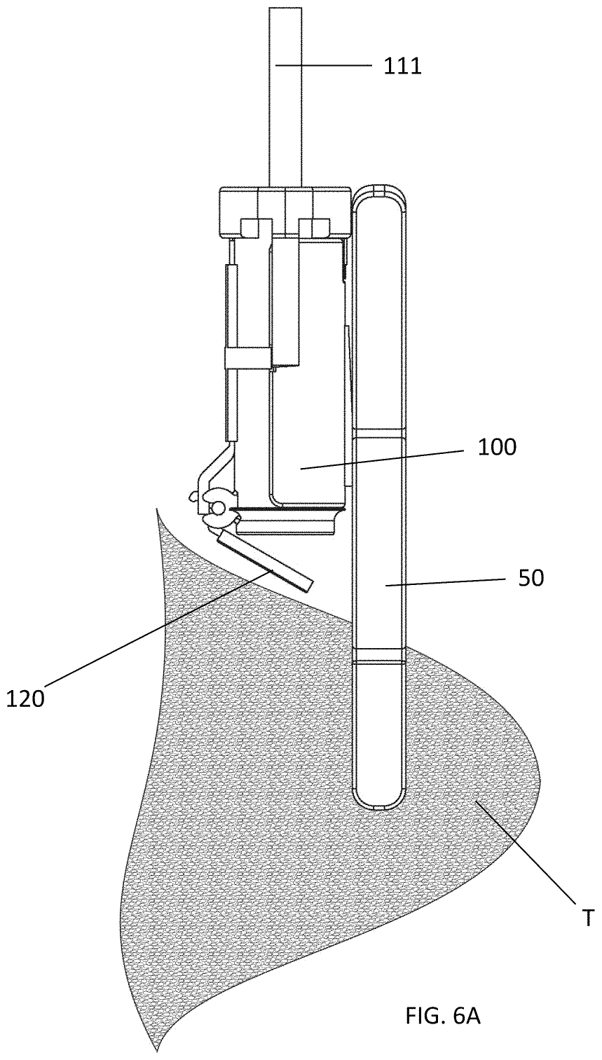

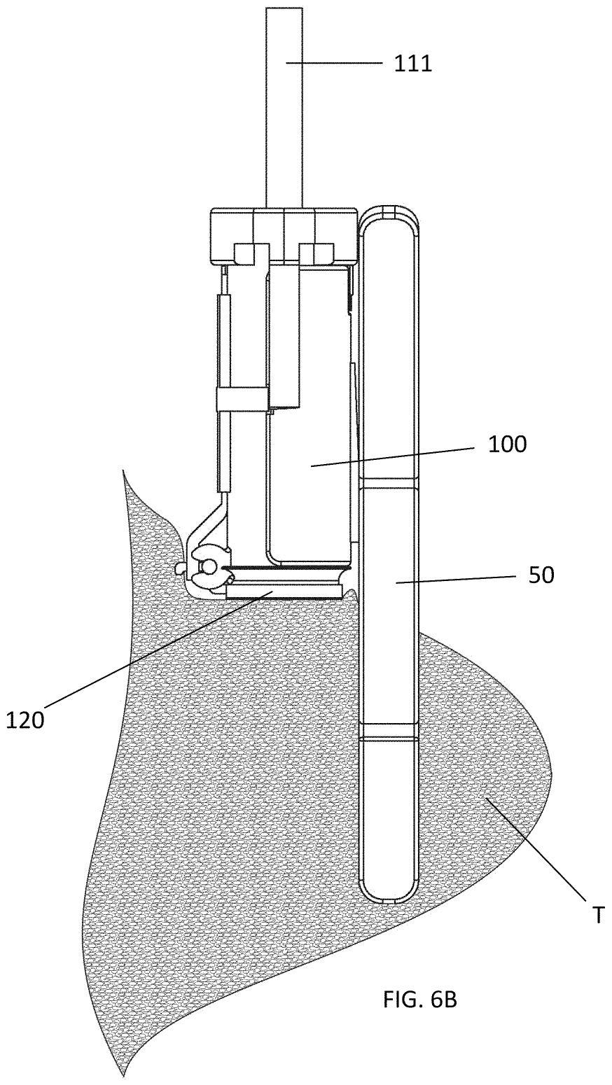

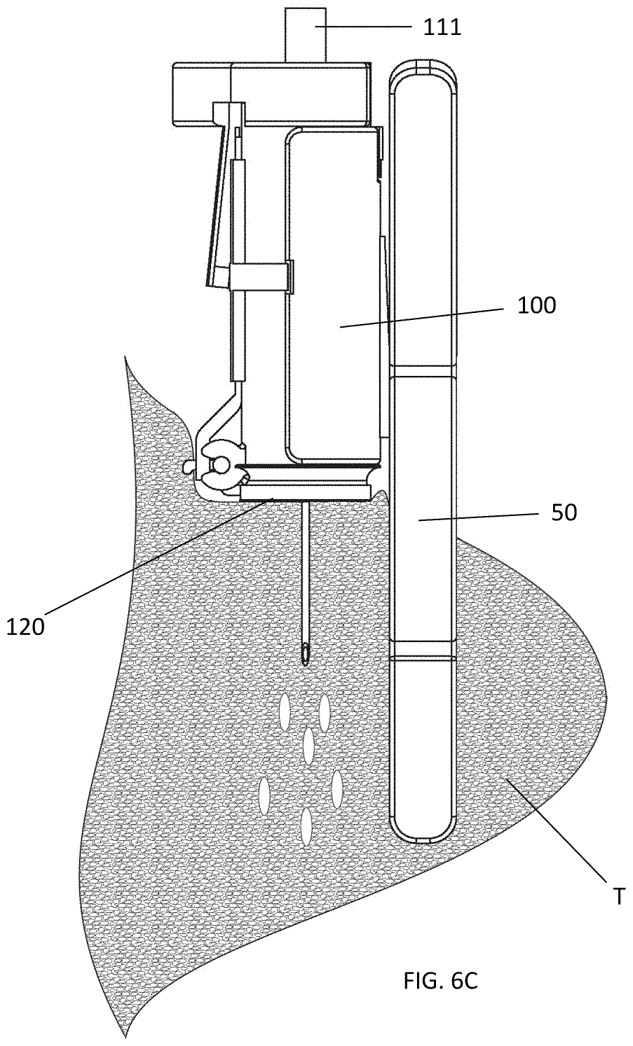

Hinged plate 120 can rotate at hinge 131 (FIG. 9B) between open and closed positions with respect to delivery end 122. When open (shown in FIG. 6A), arm 130 locks into recess 132 thereby preventing trigger 104 from rotating (FIG. 9C). When closed against a tissue fold (T, FIG. 6B), arm 130 retracts out of recess 132 thereby releasing the lock on trigger 104 and allowing manual rotation thereof (provided tissue puncturing device is correctly attached to cradle 56 as described above). This enables triggering of needle 110 release into tissue fold (T) as is shown in FIG. 6C.

Plate 120 can also include a hole alignable within a needle delivery hole in distal end 122. This can provide an additional safety mechanism for ensuring that incomplete closure of plate 120 blocks needle deployment.

Alternative tissue fold safety mechanisms can include force or distance sensors on jaws 52 or arms 54 or a bag/balloon that functions as a hydraulic pressure sensor to actuate release of trigger 104 when a predetermined hydraulic pressure is achieved. Such a hydraulic pressure sensor can be shaped as a torus positioned at the distal end of tissue penetrating device 100 with a tube connecting the inner volume of the torus to an actuator located at trigger 104.

As is mentioned hereinabove, tissue puncturing device 100 include two springs 160 and 180 for separately actuating deployment and retraction of needle 110. FIGS. 7A-C illustrate the arrangement and functions of such springs.

On initial setup, needle 110 and attached abutment 111 (also shown in FIGS. 1-2) are sequestered within subassembly 166 positioned within device body 102. Needle 110 and attached abutment 111 are under tension of spring 160 (pushing against assembly 111). As is described hereinabove, closure of plate 120 against a tissue fold (FIG. 7A) enables release of spring 160 to push abutment 111 and attached needle 110 forward (distal direction) to thereby deploy needle 110 out of delivery end 122 of tissue puncturing device 100 (FIG. 7B). Abutment 111 and attached needle 110 slide within subassembly 166 to a stop. Subassembly 166 is positioned under tension of second spring 180. Rotation of trigger 104 to the needle retraction position releases spring 180 to push backwards (proximally) subassembly 166 and enclosed needle 110 and abutment 111 (FIG. 7C).

Thus, at the end of the needle deployment and retraction cycle both springs are released and tissue puncturing device 100 cannot be reused. Tissue puncturing device 100 can include a mechanism for locking the needle in the deployed position and preventing reuse. For example, turning of trigger 104 to a final position (FIGS. 7D-F) slides element 104 into slot 537 thereby splaying out flexible elements 541 and locking element 104 in position. Slot 535 includes a narrower section 536 that transitions into a wider section 537. When the needle is deployed, flexible elements 541 are pressed inward and travel passed 536 with rounded edge 5412 sliding against slope 5362. When the needle is fully deployed, flexible elements 541 expand back and lock (at 537). This prevents needle resetting and reuse of issue puncturing device 100.

In order to facilitate delivery of fluids from a fluid source (e.g. saline bag), abutment 11 of needle 110 includes a fluid line connector 190 (FIG. 7A). Such a connector can be a Luer lock or any other connector suitable for fluid line connection. Connector 190 is in fluid communication with needle 110 through conduit 192. Distal end of needle 110 can be closed off prior to deployment ensuring that fluid does not leak out of needle 110 prior to tissue penetration.

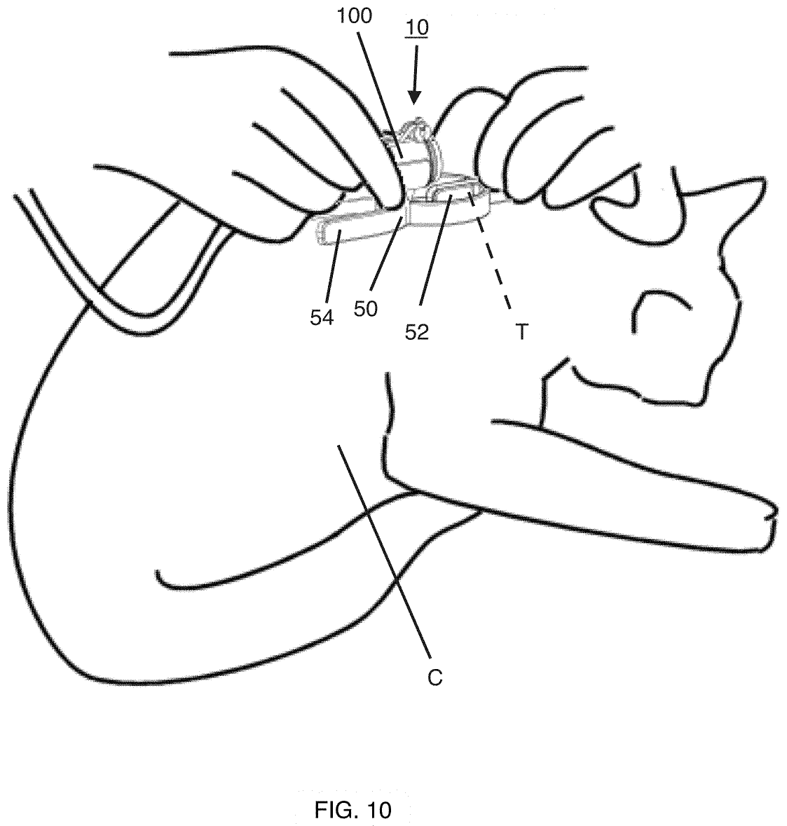

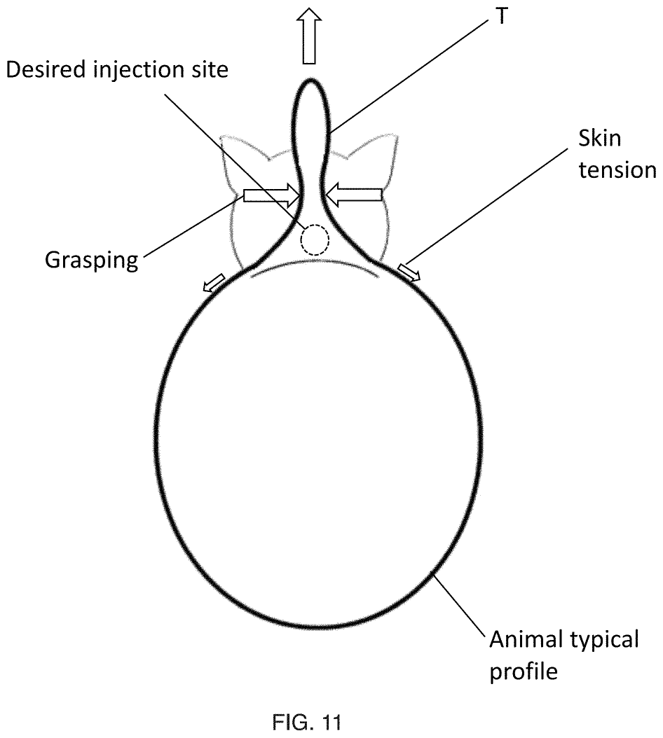

As is mentioned hereinabove, system 10 of the present invention can be used to deliver fluid (rehydration or medicament) to a mammalian subject. FIG. 10 illustrates delivery of subcutaneous infusion to a cat for the treatment of dehydration. FIG. 11 illustrates the tissue fold injection site.

Such delivery is effected as follows. Tissue clip 50 is secured by opening jaws 52 using arms 54 and pinching a tissue fold (T) on the neck/back of the cat (C) between jaws 52. Tissue clip 50 can be configured to mimic the skin fold grabbing in a kitten by its mother i.e., the pressure applied by tissue clip 50 and the size of the skin fold can be similar to that grabbed by the cat or 0.1-1 N/cm².

Tissue puncturing device is then connected to cradle 56 of tissue clip 50 while aligning tabs on tissue puncturing device with cutouts 57 and 61 on cradle 56. Correct attachment releases trigger 104 to rotate as described hereinabove. Once attached and armed, a fluid delivery line connected to a source of fluid (e.g. saline) is connected to connector 190 and trigger 104 is rotated to release needle 110 and deliver the fluid to the subcutaneous pocket within the skin fold. Following delivery (100 ±50 ml of saline over 10-20 minutes), trigger 104 is further rotated to retract needle 110 out of the tissue fold and system 10 is detached from the cat and discarded.

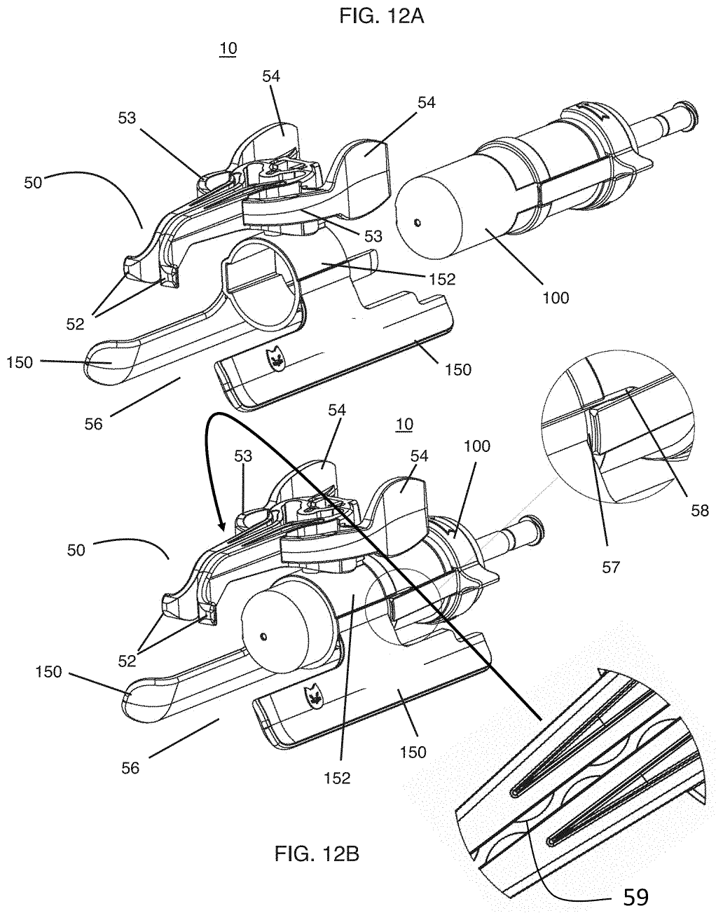

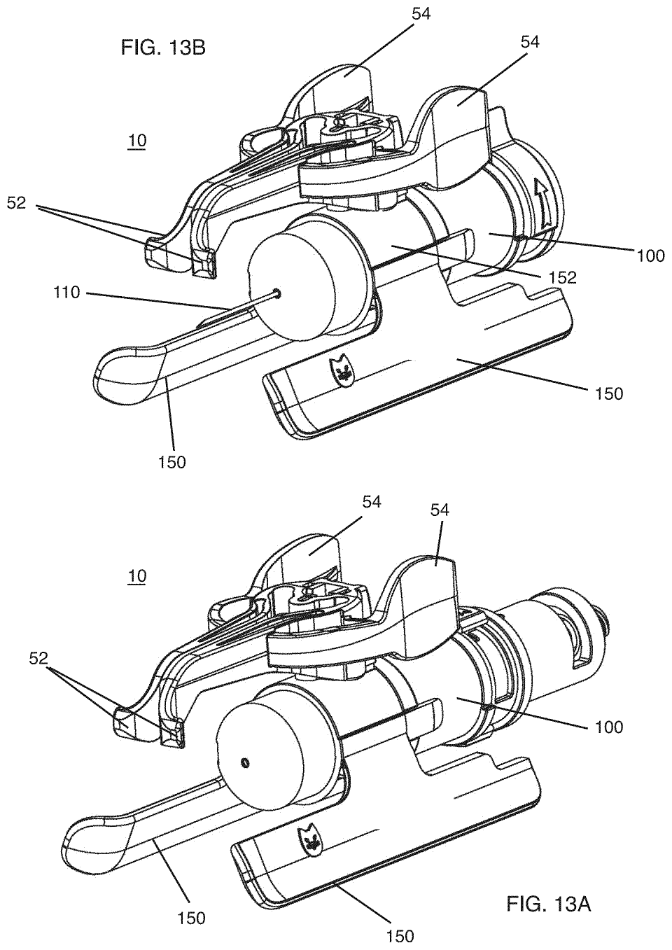

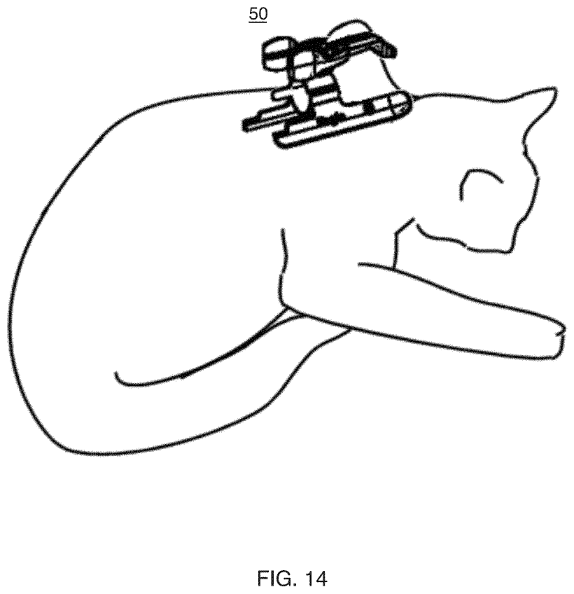

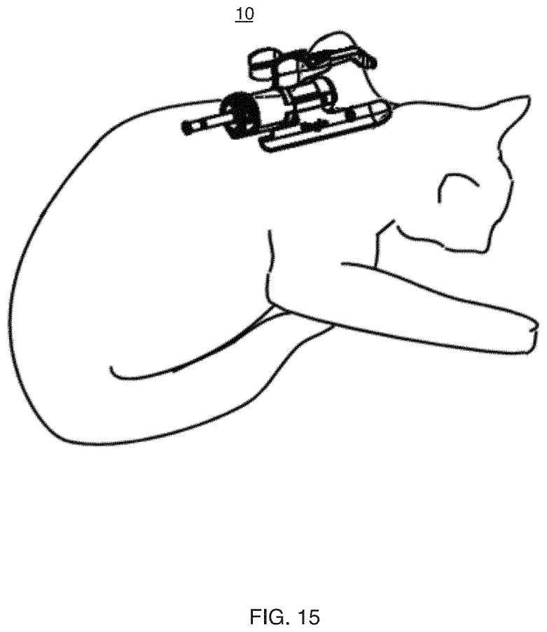

FIGS. 12A-B illustrate another embodiment of system 10. System 10 includes tissue clip 50 and tissue puncturing device 100 shown in the unattached state in FIG. 12A and attached in FIG. 12B. Deployment of a needle 110 from tissue puncturing device 100 is shown in FIGS. 13A-B. System 10 mounted on a skin fold of a cat is shown in FIGS. 14-15.

Tissue clip 50 includes jaws 52 that are connected to sprung arms 54 via connectors 53. Manually squeezing arms 54 opens jaws 52, release of arms 54 closes jaws 52 and enables grasping of a tissue fold therein. Tissue clip 50 can be fabricated from an elastic polymer such as medical grade Acetal or Polycarbonate or a metal alloy such as stainless steel.

Tissue clip 50 can be 50-120 mm in length with a maximum opening distance between jaws 52 of 1-15 mm. A tissue fold captured by jaws 52 can be 1-5 mm in thickness and 10-60 mm in height (above jaws 52). The force applied by Jaws 52 can be 0.1-1 N/cm².

Tissue clip 50 further includes a cradle 56 for engaging tissue puncturing device 100. This embodiment of tissue clip 50 includes a cradle that is positioned below jaws 52. As such, when cradle is positioned against the skin surface, jaws 52 are approximately 10-30 mm above the original skin surface. Cradle 56 includes two arms 150 attached to a cylindrical base 152 for accepting tissue puncturing device 100. Arms 150 act as skates for positioning tissue clip 50 against the tissue and for trapping the base of the tissue fold therebetween. Cradle 56 includes cutout 57 and indent 58 for correctly aligning with respective elements on tissue puncturing device 100 and allowing release of a first safety mechanism (described above). Connectors 53 include bulges 59 (see F16 attached) added to increase trapping without increasing of pressure on tissue site. This configuration of tissue clip 50 tents the tissue fold (FIG. 11) and enables tissue penetration below the region of tissue grasping by the jaws (i.e., in the space between jaws 52 and arms 150). Experiments conducted by the present inventors have shown that such a tissue grasping configuration mimics natural cat grabbing and thus calms the treated cat.

Tissue puncturing device 100 includes a device body 102 having a rotational trigger 104 disposed thereupon. Rotational trigger 104 is as described above with respect to the embodiment of system 10 shown in FIGS. 1-5.

The present system can be used as follows. The user (pet owner) creates a skin fold using the thumb and index finger of one hand. The user then slides tissue clip 150 over the skin fold using the other hand (arms 150 slide around the base of the tissue fold and jaws 52 over the tented region). The user can slide jaws 54 around the fold even if the arms 54 are not squeezed or are partially squeezed (the jaws will open over the fold). The ends of arms 150 and jaws 52 are rounded so as to not hurts the animal when the tissue clip is slid into place. The user can then mount tissue puncturing device 100 into tissue clip 50 while holding tissue clip 50 in one hand and puncturing device 100 in other hand and deploy the tissue puncturing device. An infusion source can then be connected to the tissue puncturing device port.

As used herein the term “about” refers to ±10%.

Additional objects, advantages, and novel features of the present invention will become apparent to one ordinarily skilled in the art upon examination of the following examples, which are not intended to be limiting.

### EXAMPLES

Reference is now made to the following example, which together with the above descriptions, illustrate the invention in a non-limiting fashion.

### Prototype Testing

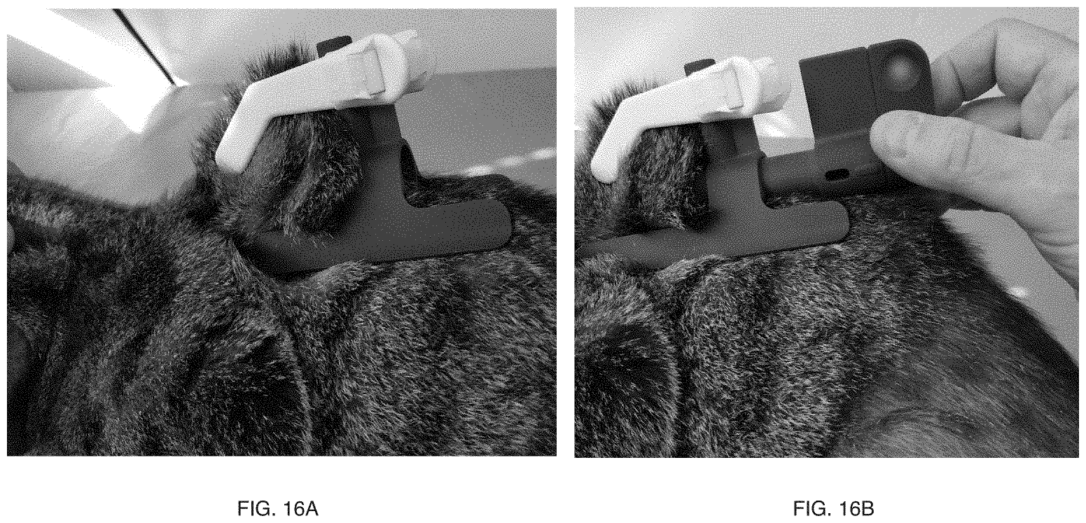

A prototype tissue clip (FIG. 16A) and attachable dummy tissue puncturing device (FIG. 16B, shown attached to clip) were fabricated using stereo lithography (SLA) of TAURUS (www(dot)materialize(dot)com/en/manufacturing/materials/taurus), a material that combines high stiffness and strength, 3D stability and reasonable flexibility.

The prototype was fabricated in order to assess the usability of a tissue clip and determine its ability to stably hold a tissue fold and direct a tissue puncturing device (injector) into the tissue fold. The recommended injecting method was mimicked by the tissue clip.

The grasping force and tissue fold size as well as the accessibility of the tissue fold to the injector were assessed using a caliper, ruler and dynamometer. Several iterations of the present design were tested and optimized for tissue fold size, stability and well as ease of use. Needle penetrating forces were not considered and the test cat was not injected. However, known values for typical penetration forces were taken into account when designing the tissue clip and tissue penetrating device.

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable subcombination.

Although the invention has been described in conjunction with specific embodiments thereof, it is evident that many alternatives, modifications and variations will be apparent to those skilled in the art. Accordingly, it is intended to embrace all such alternatives, modifications and variations that fall within the spirit and broad scope of the appended claims. All publications, patents and patent applications mentioned in this specification are herein incorporated in their entirety by reference into the specification, to the same extent as if each individual publication, patent or patent application was specifically and individually indicated to be incorporated herein by reference. In addition, citation or identification of any reference in this application shall not be construed as an admission that such reference is available as prior art to the present invention.

In addition, any priority document(s) of this application is/are hereby incorporated herein by reference in its/their entirety.

1. A system for delivering a fluid to tissue comprising
(a) a tissue clip having a pair of jaws for securing a tissue fold from a tissue surface; and
(b) a tissue puncturing device attachable to said tissue clip and being configured to deliver a tissue puncturing element into said tissue fold below said pair of jaws.

2. The system of claim 1, wherein said tissue clip includes a cradle attached to said pair of jaws.

3. The system of claim 2, wherein said cradle includes a pair of arms for resting against said tissue surface.

4. The system of claim 3, wherein said tissue puncturing element is deployable into said tissue between said pair of jaws and said arms.

5. The system of claim 1, wherein said tissue puncturing device delivers said tissue puncturing element parallel to said tissue surface when said tissue clip secures said tissue fold.

6. The system of claim 1, wherein said tissue puncturing element is not deployable when said tissue fold is not secured within said tissue clip.

7. The system of claim 1, wherein said tissue puncturing device is connectable to source of infusion.

8. The system of claim 1, further comprising:
(c) a mechanism for sensing said tissue fold and releasing a safety mechanism in said tissue puncturing device so as to enable manual deployment of a said tissue puncturing element from said tissue puncturing device.

9-18. (canceled)

19. The system of claim 8, wherein said mechanism for sensing said tissue fold includes a hinged plate capable of abutting a delivery end of said tissue puncturing device when said tissue fold is present.

20. The system of claim 19, wherein said plate covers said delivery end of said tissue puncturing device and includes an opening alignable with a delivery port of said tissue puncturing element.

21-24. (canceled)

25. A method of delivering a fluid into a skin fold comprising:
(a) securing the skin fold from a tissue surface within a tissue clip;
(b) attaching a tissue puncturing device to said tissue clip thereby deploying a mechanism for sensing said tissue fold and releasing a safety mechanism in said tissue puncturing device;
(c) deploying a tissue puncturing element from said tissue puncturing device into the skin fold.

26. The method of claim 25, wherein said tissue puncturing device is in fluid communication with a source of fluid.

27. (canceled)

28. The method of claim 25, wherein the skin fold is secured within a pair of jaws displaced from said tissue surface.

29. The method of claim 28, wherein said tissue puncturing element is deployed into a region of the skin fold between said pair of jaws and said skin surface.
